SEQUENCE LISTING (9 sequences)

SEQ ID NO: 1 - DNA, 620 bp, Mycobacterium avium subs. paratuberculosis
ggatctcggccccgatagctttcctctccttcgtcaccaactggcgcgggtccaggaacgcttggcactcgtcaatcaccacgaacaccagcggaacctgcggcgtcgggccgcgatcccaaaagttggacgatccgaatatgttgttgccctgtctaattcgatcacggactagaccggtcgcgtcattcagaatcgctgcaatctcaggcagctccagatcgtcattcatgaatcggcagctacgagcacgcaggcattccaagtcctgaccacccttcccgtcgatgacagcgaactggaccgccgctgacgcaccgaacgaccccagagcactcgtcagccacgcggttttccccgaccctggtacgcctcccactacaacgcccgatacgttcgccaacgggagcgactggtagacgcccatttcatcgatacccaaactcagagaccacgagtgggcggctggcacagacgaccattcaagcggcgaactaagcggatcgacaattctcagctgcaactcgaacacacctgggaccggctcggtgacaacaacattcggtacaccaatagctgacgccagcttcggagccgccacctgccactgtctgagatcc

SEQ ID NO: 2 - DNA, 19 bp, Artificial Sequence
  Primer
ttggacgatccgaatatgt

SEQ ID NO: 3 - DNA, 16 bp, Artificial Sequence
  Primer
agtgggaggcgtacca

SEQ ID NO: 4 - DNA, 17 bp, Artificial Sequence
  Primer
cggagacggtcacagct

SEQ ID NO: 5 - DNA, 16 bp, Artificial Sequence
  Primer
ttgcatgcctgcaggt

SEQ ID NO: 6 - DNA, 18 bp, Artificial Sequence
  DNA-section of a probe hybridizing with MAP- specific sequences
cacgcaggcattccaagt

SEQ ID NO: 7 - DNA, 18 bp, Artificial Sequence
  DNA-section of a probe hybridizing with MAP- specific sequences
tgaccacccttcccgtcg

SEQ ID NO: 8 - DNA, 23 bp, Artificial Sequence
  DNA-section of a probe hybridizing with MAP- specific sequences
gcaaggcgattaagttgggtaac

SEQ ID NO: 9 - DNA, 21 bp, Artificial Sequence
  DNA-section of a probe hybridizing with MAP- specific sequences
cagggttttcccagtcacgac

(19) United States
(12) Patent Application Publication
(10) Pub. No.: US 2008/0145848 A1
(43) Pub. Date: Jun. 19, 2008

(54) Method and Kit For Detecting Mycobacterium Avium Subsp. Paratuberculosis (Map) In Samples of Faeces, Body Tissues or Milk

(75) Inventors: Roger Stephan, Dietwil (CH); Taurai Tasara, Zurich (CH)

Correspondence Address: RANKIN, HILL & CLARK LLP, 38210 Glenn Avenue, WILLOUGHBY, OH, 44094-7808, US

(73) Assignee: UNIVERSITAT ZURICH, Zurich (CH)

(21) Appl. No.: 11/813,231

(22) PCT Filed: Jan. 6, 2006
(86) PCT No.: PCT/EP06/00074

§ 371 Date: Jul. 25, 2007

(30) Foreign Application Priority Data
Jan. 12, 2005 (DE) 10 2005 001 788.6

(51) Int. Cl. C12Q 1/68

(52) U.S. Cl. 435/6

(57) ABSTRACT

Method for the detection of Mycobacterium avium subsp. paratuberculosis (MAP) in fecal, body tissue or milk samples, in which a DNA extract is obtained from the samples, the DNA extract is processed by means of PCR in the presence of MAP-specific primers (MAP primers), and it is tested whether or not MAP-specific gene sequences were amplified in the PCR, as well as a kit for carrying out the method.

The present application includes a sequence listing, which is hereby incorporated by reference. The sequence listing was submitted using EFS-web as a text file entitled SCH17395SEQ.txt. The file was created on Jun. 25, 2007 at 2:47 p.m. and contains 4 KB of data. The file contents comply with the American Standard Code for Information Interchange (ASCII) and can be viewed using an IBM-PC compatible computer using the MS-Windows operating system.

The invention relates to a method and a kit for the detection of Mycobacterium avium subsp. paratuberculosis (MAP) in fecal, body tissue or milk samples. In particular, the invention relates to the assaying of sample material from ruminants, and more particularly to the assaying of milk or milk products made therefrom. However, the invention is also to comprise the assaying of sample material from other animal sources and from humans. Suitable tissues aside from feces and milk, that can be assayed within the scope of MAP detection include, e.g., blood, lymph tissue as well as muscle tissue, to name but a few examples.

MAP are considered to be the causative pathogen of paratuberculosis which is a chronic intestinal inflammatory disease in ruminants, in particular in cows that is common throughout the world.

It has been evident that MAP can be isolated from milk of animals diseased by clinical and subclinical paratuberculosis. Moreover, it has also been evident that MAP has higher thermal resistance as compared to other mycobacteria such that the conventional pasteurization of milk may not lead to the destruction of MAP. And lastly it has been shown that MAP was also isolated from the intestinal tissue and the blood of patients suffering from Morbus Crohn such that a causal relationship between MAP and Morbus Crohn cannot be excluded.

Especially in terms of milk hygiene, there is an urgent need for earliest possible and effective assaying and separation of diseased animals from the production cycle.

It is convenient to assay milk directly using suitable diagnostic methods. For this purpose, e.g., a method is known thus far, in which a DNA insertion sequence that is present in MAP and has been named IS900 is detected by means of PCR. The method is problematic because other mycobacteria obviously also contain IS900 elements such that this detection method is associated with a risk of samples that are not contaminated by MAP yielding false positive results.

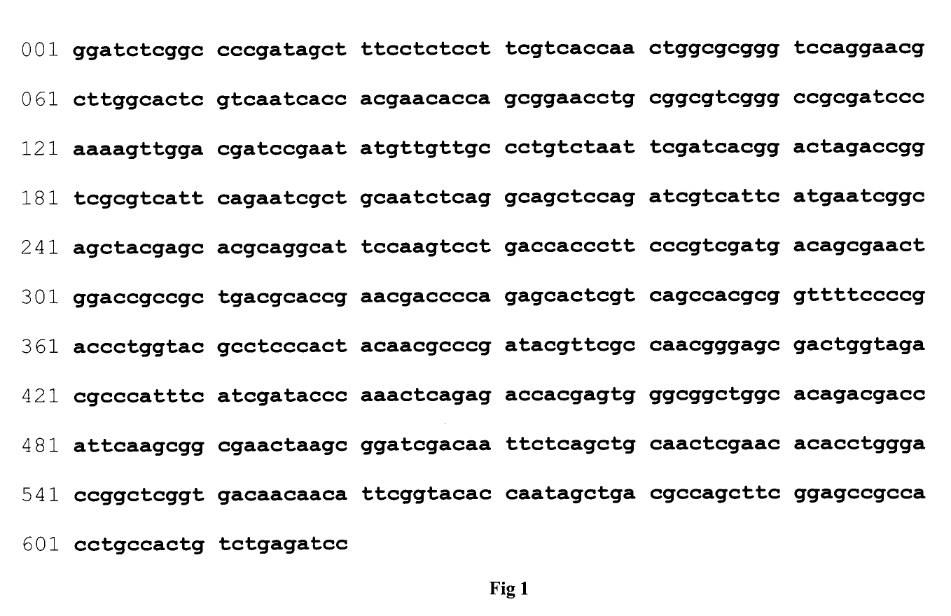

For this reason, additional gene sections of MAP have been increasingly evaluated recently for their selective utility in PCR detection systems. In this regard, Vansnick et al. described the use of primers that bind to a specific MAP sequence that was named f57 (and is described in more detail in that publication) in Vet Microbiol. 2004;100;197-204. For details of the sequence, reference shall be made to FIG. 1 and SEQ ID NO 1 which reflect the section 1-620 of the f57 gene.

However, a suitable pair of primers is only a part of a serviceable detection system. Another problem is that the samples that are usually analyzed, such as, e.g., feces or blood, but in particular milk samples, are generally only relatively difficult to assay by means of PCR since they contain a number of substances with inhibitory properties.

It was therefore the object of the invention to create a method and a kit for the detection of MAP that may be present in samples, in which all steps, or components, are optimally matched to suit each other.

This object is met by a method according to the method and kit according to the invention.

As mentioned above, the materials assayed using the method or kit according to the invention can be fecal, body tissue or milk samples that can originate in particular from ruminants, but also from other animals or from humans. Tissues other than feces or milk that are suitable as a matter of principle and can be assayed for MAP detection include, e.g., blood, lymph tissue as well as muscle tissue.

All sample materials specified present essentially similar problems with regard to their processing. In order to avoid repetition, the invention shall be described in the following in an exemplary fashion with regard to the assaying of milk from ruminants. It is to be understood, though, that the steps described can also be applied to the assaying of other materials, possibly after minor adjustments, and facilitate the desired detection in this case as well.

A DNA extract is obtained in a first step of the method according to the invention from the sample to be assayed, whereby said extract can then be further processed by means of PCR.

For this purpose, it is common to centrifuge the milk and pellet the bacteria present therein. Using other sample materials, it may be necessary to first produce a suspension and subsequently separate any coarse particles before a bacterial pellet can be obtained by means of centrifugation. Obtaining the bacteria contained in the various sample materials is a routine procedure for an expert in this field.

In a next step, the bacterial pellet obtained, e.g. from the milk sample described herein in an exemplary fashion, is lysed under lysis conditions. The conditions are selected such that mycobacteria will lyse, and usually the conditions are consistent for samples differing in origin.

One embodiment of the invention provides for further extraction and purification of the DNA, in particular by means of alcoholic precipitation, possibly to follow after previous precipitation of the proteins.

In this context, a kit and the corresponding processing protocol of GENIAL (All-tissue DNA-extraction kit, cat. no. D0502000) have proven to be particularly well-suited for this purpose.

According to a second development, the milk, or the sample of milk product, was processed using a “high pure template preparation kit, cat. no. 1796 828” of Roche Diagnostics according to the prescribed protocol. As before, an initially enzymatic treatment of the pelleted bacteria followed after the centrifugation. In addition to the procedure prescribed in the kit protocol, bacterial lysis was supported by mechanical treatment of the cells. Subsequently, the lysate was applied to a DNA-binding column and the column was then eluted as prescribed in the kit. The eluate was then further processed in the PCR in which it served as DNA extract.

With regard to the details of the methods used preferably, please refer to the corresponding protocols of the kits mentioned which are accessible to an expert in this field.

In the next step of the method according to the invention, the DNA extract thus obtained is processed in the presence of MAP-specific primers. Particularly well-suited for this purpose are primers that recognize, e.g., the f57 sequence in MAP.

A pair of primers having the following sequences is particularly preferred:

| | |
|---|---|
| SEQ ID NO 2: | TTG GAC GAT CCG AAT ATG T |
| and | |
| SEQ ID NO 3: | AGT GGG AGG CGT ACC A |

The primers mentioned correspond to sections 126-144 (SEQ ID NO 2) and 365-380 (SEQ ID NO 3) of the MAP f57 sequence. Details of these primers are summarized in Table 1a.

TABLE 1a

| | Primer | | | |
|---|---|---|---|---|
| Target | (oligonu- | Sequence | | Amplicon |
| sequence | cleotides) | (5′-3′) | Position | (bp) |
| MAPf57 | MAPf57p1 | TTG GAC GAT | 126-144 | 254 |
| (accession: | (SEQ ID | CCG AAT ATG | | |
| X70277) | NO 2) | T | | |
| | MAPf57p2 | AGT GGG AGG | 365-380 | |
| | (SEQ ID | CGT ACC A | | |
| | NO 3) | | | |

The pair or primers described here that was newly designed by the authors of the present application has been shown to recognize MAP exclusively and no other mycobacterial strains in a highly specific fashion. The pair of primers has been tested in PCR samples containing MAP and other mycobacterial strains, whereby the specificity for MAP of the primers thus developed as well as the suitability of f57 as a specific target for the detection of MAP was confirmed (Tasara et al. Int. J. Food Microbiol., submitted).

False positive results can therefore be almost completely excluded through the use of the primers mentioned above.

However, possible false negative results are another problem.

As mentioned above, milk, similar to the other sample materials mentioned, is a difficult sample material because of the presence of PCR inhibitors therein. Likewise, in the methods for obtaining a DNA extract, whose use is described above to be particularly preferred, it cannot be excluded that the PCR sample may possibly still contain substances that inhibit the PCR.

In a preferred embodiment, the invention therefore provides the PCR to be carried out in the presence of an internal amplification control.

In this context, suitable amplification controls can be any target DNA sequences (control DNA) that can be amplified in a standardized and reproducible fashion under similar conditions as the MAP sequences using correspondingly designed primers (control primers).

It is particularly preferred to carry out the internal amplification control in the same sample that is used for detection of the MAP sequence. In this case, it is necessary to ensure that the amplification products of the control DNA and MAP sequence can be distinguished from each other.

In the scope of the invention, it is preferred to use PuC19 plasmid DNA as control DNA. Details of this plasmid, in particular with regard to its sequence, have been published, e.g., by Yanisch-Perron et al. in Gene 33(1), 103-119 (1985). PuC19 can be obtained from ATCC (ATCC 37254/L09137) or from New England Biolabs (cat. no. N3041S).

Suitable control primers can have, e.g., the following sequences:

| | |
|---|---|
| SEQ ID NO 4: | CGG AGA CGG TCA CAG CT |
| SEQ ID NO 5: | TTG CAT GCC TGC AGG T |

The primers mentioned above hybridize to sections 49-65 (SEQ ID NO 4) or 433-448 (SEQ ID NO 5) of the PuC19 plasmid DNA mentioned above. Details of these primers are summarized in Table 1b.

TABLE 1b

| Target | Oligonu- | Sequence | | Amplicon |
|---|---|---|---|---|
| sequence | cleotides | (5′-3′) | Position | (bp) |
| PuC19 | PuC19fw | CGG AGA CGG | 49-65 | 400 |
| plasmid DNA | (SEQ ID | TCA CAG CT | | |
| (accession: | NO 4) | | | |
| L09137) | PuC19rv | TTG CAT GCC TGC AGG T | 433-448 | |
| | (SEQ ID | | | |
| | NO 5) | | | |

The developments of the method according to the invention described thus far allow even tiny amounts of MAP, e.g. in milk, to be detected very reliably. Studies of the applicant have shown that as few as 10 MAP cells per ml of milk can thus be detected.

The time factor is another problem. Usually, the results of assays on milk samples need to be available relatively rapidly for logistic reasons. However, using conventional PCR and subsequent gel analysis it takes 4-6 hours from sampling to having the results available, which usually will be too long.

An advantageous development of the invention therefore provides for the processing of the DNA extract to be by real-time PCR.

Real-time PCT methods have been known for some time and differ from conventional PCR methods essentially in that the PCR sample, in addition, contains probes that generate a signal that changes in correlation with the increase of amplified DNA. Moreover, the PCR apparatus employed (PCR cycler) comprises a measuring facility that detects the signals thus generated in the samples during the PCR, i.e. it can be detect instantaneously, i.e. during the ongoing measurement, whether or not amplification of the target sequence occurred.

Probe and cycler systems equipped with corresponding measuring facilities that are suitable for real-time PCR are being marketed by various manufacturers that are known to the expert in this field.

One known system of this type is, e.g., the Sybr-Green system. Sybr-Green is a dye that intercalates into double-strand DNA and generates a fluorescence signal that can be measured in a suitable cycler only when it is in the intercalated state. The signal gets stronger the more double-strand DNA is present into which the dye can intercalate. One problem of the Sybr-Green system mentioned is that it allows only for non-specific detection of double-strand DNA.

Therefore, it is particularly preferred for the invention to provide for the use of specific probes for detection of the amplification products generated during the PCR, whereby said specific probes generate a signal only upon amplification of MAP sequences and/or of the internal control sequence. It is self-evident that it must be feasible to differentiate the signals generated when the amplification control is processed in the same sample from the signals generated upon amplification of the MAP sequence, which can be effected with no difficulty by adequate selection of markers.

Specific probes usually comprise a DNA section that is selected such that it can hybridize specifically to a section of the target DNA. A marker or marker system is coupled to said DNA section and generates a different signal upon hybridization than in the non-hybridized state. Suitable probes have become known amongst experts, e.g. by the name of “TAQMAN probes”.

It is particularly preferable in the method according to the invention to use a different system, i.e. the so-called Light-Cycler System of Roche-Diagnostics. This systems works with 2 probes per each target which both hybridize to neighboring sections of the target sequence. One of the probes is fluorescine-labeled, whereas the other is labeled with a fluorescence dye, e.g. LC-Red-705 or LC-Red-640 (Roche-Diagnostics). If close spatial proximity is established, as is the case after hybridization of the two probes to the target DNA, the fluorescine can activate the fluorescence dyes which then generate a signal of the specified wavelength, i.e. 705 nm or 640 nm.

MAP probes having the following sequences have proven particularly useful in the method according to the invention:

| | |
|---|---|
| SEQ ID NO 6: | CAC GCA GGC ATT CCA AGT |
| and | |
| SEQ ID NO 7: | TGA CCA CCC TTC CCG TCG |

Probes having the following sequences have been determined to be particularly preferred for the internal amplification control:

| | |
|---|---|
| SEQ ID NO 8: | GCA AGG CGA TTA AGT TGG GTA AC |
| SEQ ID NO 9: | CAG GGT TTT CCC AGT CAC GAC |

Details of the probes mentioned are summarized in Table 1c:

TABLE 1c

| | Probe | | |
|---|---|---|---|
| Target | oligonu- | | |
| sequence | cleotides | Sequence (5′-3′) | Position |
| MAPf57 | MAPf57- | CAC GCA GGC ATT CCA | 250-267 |
| (accession: | 3′Fluo | AGT | |
| X70277) | (SEQ ID | | |
| | NO 6) | | |
| | MAPf57- | TGA CCA CCC TTC CCG | 270-287 |
| | 5′Red705 | TCG | |
| | (SEQ ID | | |
| | NO 7) | | |
| PuC19 | PuC19- | GCA AGG CGA TTA AGT | 330-350 |
| plasmid DNA | 3′Fluo | TGG GTA AC | |
| (accession: | (SEQ ID | | |
| L09137) | NO 8) | | |
| | PuC19- | CAG GGT TTT CCC AGT | 355-375 |
| | 5′Red640 | CAC GAC | |
| | (SEQ ID | | |
| | NO 9) | | |

Moreover, the invention relates to a kit that contains the reagents required to carry out the method.

The kit according to the invention contains at least reagents for obtaining a DNA extract from mycobacterial cells that may be present in sample material, in particular in milk or milk products, and reagents for carrying out a real-time PCR, containing a pair of primers that is specific for a MAP sequence, an internal amplification control as well as probes that hybridize to the MAP sequence and internal amplification control and each comprise different detectable markers.

In addition, the kit can contain a positive control for the PCR. This can, e.g., be the above-mentioned MAP sequence, f57 (SEQ ID NO 1), cloned into a suitable vector for processing in a separate PCR sample in order to ensure that the selected PCR conditions are in fact suitable for the amplification and detection of MAP sequences.

The invention shall be illustrated in detail in the following by means of some examples.

### EXAMPLE 1

Sample treatment and DNA extraction using the “High pure template preparation kit”:

100 μl Triton X-100 (Calbiochem) are added to 10 ml of milk and centrifuged at 4,500 rpm for 30 min. The pellet thus obtained is resuspended in 0.5 ml of the supernatant and transferred to an Eppendorf tube. A second centrifugation is carried out at 14,000 rpm for 10 min. Then the supernatant is discarde. The obtained pellets are resuspended in 240 μl MAP lysis buffer (20 mM Tris-HCL (pH 8.0), 400 mM NaCl, 0.6% SDS, 2 m MEDTA). After addition of 60 μl proteinase K the sample at 55° C. is incubated for 2 hours with periodical mixing. After addition of 300 μl buffer (Roche kit) and the glass beads the incubated sample is transferred to a Ribolyser (Hybaid, Ashford) (6.5 msec⁻¹ for 45 s). The sample is then immediately heated to 70° C. for 10 minutes in order to destroy any DNAses that may be present. Subsequently the sample is allowed to cool at room temperature for 1 min, add 150 μl isopropanol (2-propanol, Fluka), centrifuged briefly, and applied to the DNA-binding column of the kit. The DNA templates are eluted with 100 μl elution buffer (kit). 5 μl aliquots are used in the subsequent PCR.

### EXAMPLE 2

PCR conditions:

Real-time PCR is carried out in 20 μl glass capillaries using the Light Cycler 2.0 instrument (Roche Diagnostics). The reaction mixture consists of 1× LightCycler-Faststart DNA Master Plus™ hybridization probes mix (Roche Diagnostics), 800 nM of each primer (MAPf57p1/SEQ NO ID 2, MAPf57p2/SEQ NO ID 3, PuC400fw/SEQ NO ID 4 and puC400rv/SEQ NO ID 5), 200 nM of each probe, and 2,000 copies of a PuC19 plasmid DNA that is used as internal amplification control. The amplification commences with an initial pre-incubation at 95° C. for 10 minutes followed by 45 cycles (95° C. for 10 s, 56° C. for 20 s, and 72° C. for 18 s).

### EXAMPLE 3

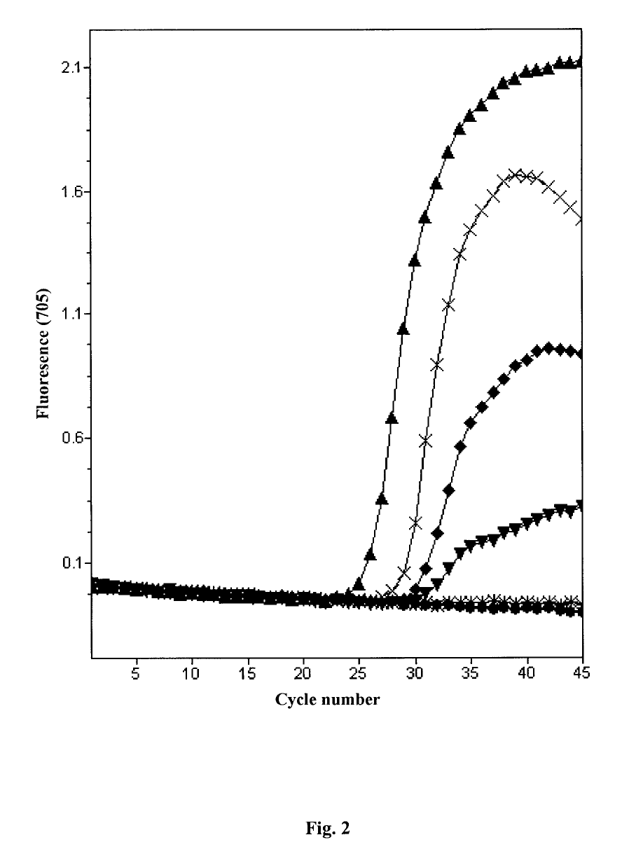

The DNA from milk samples artificially contaminated with MAP was extracted and purified according to the examples given above, and the respective DNA extracts were processed by means of real-time PCR. The results are shown in FIG. 2, in which the fluorescence measured at 705 nm (Y axis) is plotted over the number of cycles (X axis); the symbols used in FIG. 2 represent the following MAP starting concentrations:
▴=10⁴ MAP cells/ml of milk
x=10³ MAP cells/ml of milk
♦=10² MAP cells/ml of milk
▾=10¹ MAP cells/ml of milk
*=10⁰ MAP cells/ml of milk
=Negative control (milk sample that was not artificially contaminated)

The dependence of the increase in fluorescence on the initial concentration of cells is clearly evident from approx. cycle 20-30 in FIG. 2. Moreover, it is evident that a clearly detectable signal increase can still be measured even at starting concentrations of 10 MAP cells/ml of milk.

1. A method for the detection of Mycobacterium avium subsp. paratuberculosis (MAP) in fecal, body tissue or milk samples, in which
a DNA extract is obtained from the samples;
the DNA extract is processed by means of PCR in the presence of MAP-specific primers (MAP primers); and
it is tested whether or not MAP-specific gene sequences were amplified in the PCR.

2. The method according to claim 1, wherein the assayed sample originates from ruminants.

3. The method according to claim 2, wherein the assayed sample is milk or a milk product made therefrom.

4. The method according to claim 1, wherein the bacteria present in the sample are isolated in order to obtain the DNA extract, the isolated bacteria are incubated under lysis conditions, and the DNA present in the lysate is then extracted and purified.

5. The method according to claim 4, wherein the DNA extraction is effected by means of alcoholic precipitation.

6. The method according to claim 4, wherein the DNA extraction is effected by means of a DNA-binding column.

7. The method according to claim 1, wherein the MAP primers used in the PCR are specific for the f57 gene of MAP.

8. The method according to claim 7, wherein the primer sequences correspond to SEQ ID NO 2 and SEQ ID NO 3.

9. The method according to claim 1, wherein the PCR is carried out in the presence of an internal amplification control.

10. The method according to claim 9, wherein a defined control DNA sequence and control primers recognizing the control DNA sequence are used as internal amplification control.

11. The method according to claim 10, wherein PuC19 plasmid DNA is used as control DNA.

12. The method according to claim 10, wherein the control primers comprise sequences in accordance with SEQ ID NO 4 and SEQ ID NO 5.

13. The method according to claim 10, wherein the DNA extract and the internal amplification control are processed in the same PCR sample.

14. The method according to claim 1, wherein the PCR is carried out in the presence of probes that generate signals whose intensity depends on the amount of amplified DNA, and wherein the signals thus generated are measured either continuously or at different time points during the PCR.

15. The method according to claim 14, wherein probes are used that comprise a DNA section that hybridizes to MAP-specific gene sequences (MAP probes) and/or control DNA gene sequences (control probes) and a marker coupled to the DNA section, whereby said marker emits a different signal in the hybridized state of the DNA section than in the non-hybridized state.

16. The method according to claim 15, wherein the DNA sections of MAP probes comprise sequences in accordance with SEQ ID NO 6 and SEQ ID NO 7.

17. The method according to claim 15, wherein the DNA sections of control probes comprise sequences in accordance with SEQ ID NO 8 and SEQ ID NO 9.

18. The method according to claim 15, wherein the markers of MAP probes and control probes that are coupled to the DNA sections generate signals that can be differentiated from each other.

19. The method according to claim 15, wherein the markers coupled to the DNA sections are fluorescence dyes.

20. A kit for carrying out the method according to claim 1, the kit containing:
reagents for obtaining a DNA extract from mycobacterial cells that may be present in sample material, and
reagents for carrying out a real-time PCR, containing a pair of primers that is specific for a MAP sequence, an internal amplification control as well as probes that hybridize to the MAP sequence and internal amplification control and each comprise different detectable markers.
